(19) United States
(12) Patent Application Publication
(10) Pub. No.: US 2011/0054520 A1
(43) Pub. Date: Mar. 3, 2011

(54) Devices and Methods for Occluding a Fistula

(75) Inventors: Stephen E. Deal, Charlotte, NC (US); Charles W. Agnew, West Lafayette, IN (US)

(73) Assignee: Wilson-Cook Medical Inc., Winston-Salem, NC (US)

(21) Appl. No.: 12/941,363

(22) Filed: Nov. 8, 2010

Related U.S. Application Data
(62) Division of application No. 11/844,115, filed on Aug. 23, 2007.
(60) Provisional application No. 60/839,976, filed on Aug. 24, 2006.

(51) Int. Cl. A61B 17/00

(52) U.S. Cl. 606/213

(57) ABSTRACT

A method of occluding a fistula in a patient is provided. The method includes inserting a placement member having a coupling structure, such as a wire guide having a loop at one end, through the primary opening of a fistula and at least partially into the fistula tract; connecting the coupling structure to a medical device, such as a plug, graft, or other occluding member; and inserting the medical device into the fistula by pulling the placement member through the fistula until the medical device contacts the interior wall of the fistula. Medical devices and systems for occluding fistulas are also provided.

### RELATED APPLICATIONS

This application is a division of U.S. patent application Ser. No. 11/844,115, filed Aug. 23, 2007 which claims the benefit of the filing date under 35 U.S.C. §119(e) of Provisional U.S. Patent Application Ser. No. 60/839,976, filed Aug. 24, 2006, the contents of which are hereby incorporated by reference.

### TECHNICAL FIELD

The present invention relates generally to medical devices and methods and, in particular, to medical devices and methods for treating fistulas.

### BACKGROUND

A variety of abnormal passages called fistulas can occur in a mammalian body. Such fistulas may be caused by, for example, an infection, a congenital defect, inflammatory bowel disease (such as Crohn's disease), irradiation, trauma, neoplasia, childbirth, or a side effect from a surgical procedure.

Some fistulas occur between the vagina and the bladder (vesico-vaginal fistulas) or between the vagina and the urethra (urethro-vaginal fistulas). These fistulas may be caused by trauma during childbirth. Traditional surgery for these types of fistulas is complex and not very successful.

Other fistulas include, but are not limited to, tracheo-esophageal fistulas, gastro-cutaneous fistulas, fistulas extending between the vascular and gastrointestinal systems, and any number of anorectal (ano-cutaneous) fistulas, such as fistulas that form between the anorectum and vagina (recto-vaginal fistulas), between the anorectum and bladder (recto-vesical fistulas), between the anorectum and urethra (recto-urethral fistulas), or between the anorectum and prostate (recto-prostatic fistulas). Anorectal fistulas can result from infection in the anal glands, which are located around the circumference of the distal anal canal forming an anatomic landmark known as the dentate line. Approximately 20-30 such glands are found in humans. Infection in an anal gland can result in an abscess. This abscess can then track through soft tissues (e.g., through or around the sphincter muscles) and into the perianal skin, where it drains either spontaneously or surgically. The resulting void through the soft tissue is known as a fistula. The internal or inner opening of the fistula, usually located at or near the dentate line, is known as the primary opening. The primary opening is usually the high pressure end of a fistula. Any external or outer openings, which are usually located in the perianal skin, are known as the secondary openings. The secondary openings are usually the low pressure end of a fistula.

Fistulas, such as anorectal fistulas, may take various paths. Such paths vary in complexity. Fistulas that take a straight line path from the primary opening to the secondary opening are known as simple fistulas. Fistula that contain multiple tracts ramifying from the primary opening and have multiple secondary openings are known as complex fistulas.

The anatomic path that an anorectal fistula takes is classified according to its relationship to the anal sphincter muscles. The anal sphincter includes two concentric bands of muscle: the inner, or internal, sphincter and the outer, or external, sphincter. Fistulas which pass between the two concentric anal sphincters are known as inter-sphincteric fistulas. Those which pass through both internal and external sphincters are known as trans-sphincteric fistulas, and those which pass above both sphincters are called supra-sphincteric fistulas. Fistulas resulting from Crohn's disease usually ignore these anatomic paths, and are known as extra-anatomic fistulas.

Many complex fistulas contain multiple tracts, some blind-ending and others leading to multiple secondary openings. One of the most common and complex types of fistulas is known as a horseshoe fistula. In this instance, the infection starts in the anal gland (the primary opening) at or near the twelve o'clock location (with the patient in the prone position). From this primary opening, fistulas pass bilaterally around the anal canal, in a circumferential manner, forming a characteristic horseshoe configuration. Multiple secondary openings from a horseshoe fistula may occur anywhere around the periphery of the anal canal, resulting in a fistula tract with a characteristic horseshoe configuration.

One technique for treating an abnormal bodily passage such as a fistula is to occlude the passage with an occluding member, such as a plug or graft. Examples of such occluding members and related methods are disclosed in co-pending U.S. Application Publication Nos. 2005/0070759A1, published Mar. 31, 2005, 2005/0159776A1, published Jul. 21, 2005, 2006/0074447A2, published Apr. 6, 2006, and 2007/0031508, published Feb. 8, 2007, and U.S. 2007/0198059, published Aug. 23, 2007, which are hereby incorporated by reference in their entirety. Such occluding members may be pulled through the primary opening of a fistula until the occluding member is securely lodged within the fistula. The occluding member may be further secured within the fistula by the use of sutures or a cap associated with the body of the plug or graft.

Typical techniques for treating a fistula involve draining infection from the fistula tract and maturing it prior to a definitive closure or sealing procedure by inserting a narrow diameter rubber drain, known as a seton, through the tract. This is usually accomplished by inserting a fistula probe through the outer (secondary) opening and gently guiding it through the fistula, and out through the inner (primary) opening. A seton, thread or tie is then affixed to the tip of the probe, which is then withdrawn back out of the tract, leaving the seton in place. The seton may then be tied as a loop around the contained tissue and left for several weeks or months.

Another technique for treating a fistula involves the use of a plug-like closure device in combination with a drainage thread or seton, as disclosed in co-pending U.S. Publication No. 2005/0049626, published Mar. 3, 2005, which is hereby incorporated by reference in its entirety. In this technique, a closure device is provided with a flexible application string that can be used to drain secretions or other undesirable liquids from the fistula. A rod-like instrument is pushed into the fistula from the outer opening and is used to investigate the trajectory of the fistula. After the instrument is pushed forward enough to protrude from the inner opening, the application string is pulled through the fistula from the inner opening until the closure device “sticks” in the inner opening. The closure device is then pushed as far as necessary for it to be tightly secured within the fistula.

Still other techniques for treating fistulas are described in U.S. application Ser. No. 11/415,403, filed May 1, 2006; and U.S. patent application Ser. No. 11/766,606, filed Jun. 21, 2007, which are hereby incorporated by reference in their entirety.

The above techniques can be difficult for some physicians, such as endoscopists, to perform. Therefore, there remains a need for simplified procedures and new medical devices and systems for occluding fistulas.

### SUMMARY

The present invention provides devices, systems, and minimally invasive methods for occluding fistulas that overcome the shortcomings of the prior art and simplify the implantation of an occluding member in a fistula of a patient.

The present invention may be used to occlude any type of abnormal bodily passage or fistula. For example, the claimed devices, systems, and methods may be used to occlude tracheo-esophageal fistulas, gastro-cutaneous fistulas, anorectal fistulas, fistulas occurring between the vagina and the urethra or bladder, fistulas occurring between the vascular and gastrointestinal systems, or any other type of fistula.

In one aspect of the present invention, a medical device for occluding a fistula is provided. In some embodiments, the medical device comprises an occluding member body configured to be placed within a fistula and to occlude the fistula. The medical device further comprises a coupling structure such as a loop or an elongate member, which facilitates implantation of the device. The device may be made of any biocompatible material. In some desirable embodiments, the device is made of a remodelable extracellular matrix material, such as small intestinal submucosa. In various embodiments, the medical device also includes a detachable sheath covering at least a portion of the occluding member body. In one such embodiment, the coupling structure is attached to the detachable sheath.

In another aspect of the present invention, a system for occluding a fistula is provided. In some embodiments, the system comprises an occluding member including an occluding member body and a first coupling structure, as well as, a wire guide having a second coupling structure, where the second coupling structure is configured to engage the first coupling structure and to facilitate insertion of the occluding member into the fistula. In some embodiments, one coupling structure is a closed loop and the other coupling structure is a loop having a discontinuity. In other embodiments, one coupling structure is a loop and the other coupling structure is a member having an elongate shape or other shape suitable for introduction into a fistula. In other embodiments, the occluding member also includes a sheath covering at least a portion of the occluding member body. In one embodiment, the first coupling structure is attached to the sheath.

In still another aspect of the present invention, a method of occluding a fistula is provided. In some embodiments, the method comprises: (a) inserting a placement member into the primary opening of a fistula and at least partially into the fistula tract, where the placement member comprises a thin, elongated member (such as a wire guide) having a coupling structure, such as a loop, at one end; (b) attaching the coupling structure to an occluding member, such as a device including a graft, plug, or other occluding member body; and (c) inserting the occluding member into the fistula by pulling the placement member through the fistula until the occluding member body contacts the interior wall of the fistula. In some embodiments, the coupling structure is a closed loop, a loop having a discontinuity, or a member having an elongate shape or any other shape capable of being coupled to an occluding member and suitable for introduction into a fistula. The occluding member may also contain a coupling structure configured to engage the coupling structure of the placement member. In some embodiments, an endoscope is utilized to assist with insertion of the placement member into the fistula. An instrument channel within the endoscope may be used to facilitate the delivery of wire guides, catheters, medical devices, and the like into the fistula during the implantation procedure.

In one embodiment of the method of occluding a fistula, inserting the placement member into the primary opening and at least partially into the fistula tract includes (a) inserting a wire guide into the primary opening and at least partially into the fistula tract, (b) placing a catheter over the wire guide and advancing the catheter at least partially into the fistula tract, (c) removing the wire guide from the catheter and the fistula tract, (d) inserting the placement member into the catheter and advancing the placement member at least partially into the fistula tract, and (e) removing the catheter from the fistula tract.

In another embodiment of the method of occluding a fistula, the occluding member includes a sheath covering at least a portion of the occluding member body. The method includes pulling the placement member so as to detach the sheath from the occluding member body and to extract the sheath from the fistula.

Additional features and advantages of the present invention will be apparent to one of ordinary skill in the art from the drawings and detailed description of the preferred embodiments below. Moreover, it should be appreciated that several aspects of the present invention can be performed with alternative types of wire guides, catheters, endoscopes, occluding members, and other medical devices.

### BRIEF DESCRIPTION OF THE DRAWINGS

FIG. 1 shows one embodiment of a medical device coupled to a placement member;

FIG. 2 shows another embodiment of a medical device coupled to a placement member;

FIG. 3 shows still another embodiment of a medical device coupled to a placement member;

FIGS. 4-12 show successive steps of one embodiment of the method in which a medical device similar to the medical device illustrated in FIG. 1 is being implanted into an anorectal fistula;

FIG. 13 shows an alternative embodiment of the medical device including a cap, wherein the medical device is implanted within an anorectal fistula of a patient;

FIGS. 14a and 14b show another embodiment of a medical device coupled to a placement member;

FIG. 15 shows yet another embodiment of a medical device coupled to a placement member, and

FIGS. 16 and 17 show one embodiment of a method of implanting a medical device within a fistula.

### DETAILED DESCRIPTION OF THE PREFERRED EMBODIMENTS

While the present invention may be embodied in many different forms, for the purpose of promoting an understanding of the principles of the present invention, reference will now be made to the embodiments illustrated in the drawings, and specific language will be used to describe the same. It will nevertheless be understood that no limitation of the scope of the invention is thereby intended. Any alterations and further modifications in the described embodiments and any further applications of the principles of the present invention as described herein are contemplated as would normally occur to one skilled in the art to which the invention relates.

Turning now to a discussion of the particular medical devices, systems, and methods of the present invention useful for treating fistulas, illustrative medical devices of the invention are configured to block at least the primary opening of a fistula, i.e., the primary opening and potentially one or more other segments of a fistula, for example, the fistula tract and/or any secondary openings. In this context, the term “fistula tract” is meant to include, but is not limited to, a void in the soft tissues extending from a primary fistula opening, whether blind-ending or leading to one or more secondary fistula openings.

The medical devices, systems, and methods of the present invention may be used to occlude any type of fistula. For example, fistulas such as anorectal fistulas, tracheo-esophageal fistulas, gastro-cutaneous fistulas, or fistulas occurring between the vagina and bladder (vesico-vaginal fistulas), between the vagina and urethra (urethro-vaginal fistulas), between the anorectum and vagina (recto-vaginal fistulas), between the anorectum and bladder (recto-vesical fistulas), between the anorectum and urethra (recto-urethral fistulas), between the anorectum and prostate (recto-prostatic fistulas), or between the vascular and gastrointestinal systems may be treated with the devices, systems, and methods of the present invention.

Generally, the medical devices of the present invention comprise an occluding member configured for implantation into a fistula. The occluding member may have any suitable configuration, such as the configurations disclosed in co-pending U.S. Publication Nos. 2005/0049626A1, published Mar. 3, 2005, 2005/0070759A1, published Mar. 31, 2005, 2005/0159776A1, published Jul. 21, 2005, 2006/0074447A2, published Apr. 6, 2006, and 2007/0031508, published Feb. 8, 2007, U.S. patent application Ser. No. 11/415,403 (Cook Biotech Incorporated, assignee), filed May 1, 2006, U.S. 2007/0198059, published Aug. 23, 2007, and U.S. patent application Ser. No. 11/766,606, filed Jun. 21, 2007. For example, the occluding member may be of any suitable dimensions and may have a body that is generally convex, concave, S-shaped, straight, curved, flat, polygonal, conical, cylindrical, elliptical, or hemispherical, or it may have any other configuration capable of being inserted into and secured within a fistula. In certain embodiments, the body of the occluding member comprises a plug or graft having one or more lumens extending at least partially through the plug or graft body along its length. In other embodiments, the occluding member comprises a body having a central lumen to facilitate deployment of the occluding member body over a guidewire or other placement member. In some embodiments, the body of the occluding member has portions that are tapered and/or curvilinear. In other embodiments, the body of the occluding member is curved to conform to the shape of the fistula, thereby facilitating introduction of the occluding member, a secure fit of the occluding member within the fistula, and less discomfort for the patient.

The body of the occluding member of the present invention may have any dimension suitable for implantation within a fistula. The body of the occluding member has a size and shape adapted to extend into at least a portion of a fistula tract, and is generally (but not necessarily) of sufficient dimension to fill a fistula, or a segment thereof, e.g., the primary fistula opening, fistula tract, and/or any secondary fistula openings, either alone or in combination with other components of the occluding member and/or other similar or differing medical devices. The body of the occluding member may or may not be sized and shaped to fill the entire fistula tract.

In addition to an occluding member body, the medical devices of the present invention may include other components that are integrally incorporated into the medical device as a single unitary construct or configured as separate components that are associated with the occluding member body in any suitable manner. For example, a cap may be integral with, attached to, or otherwise associated with the body of the occluding member, as described in co-pending U.S. Publication No. 2007/0031508, published Feb. 8, 2007. The cap may be used to prevent unintentional displacement of the occluding member after implantation. In some embodiments, the cap is configured to contact portions of an alimentary canal wall adjacent to the primary opening of an anorectal fistula, and the body of the occluding member is configured to extend into at least a portion of the fistula tract. In other embodiments, a second cap (which may be expandable) configured to contact portions of the tissue adjacent to a secondary opening is associated with or attached to the body of the occluding member before, during, or after implantation. In still other embodiments, the medical device of the present invention also includes an elongated tail, which may be used to facilitate deployment of the occluding member and to eliminate the need for a separate seton placement step in the implantation procedure.

In certain embodiments, the medical device includes a coupling structure. The coupling structure may have any suitable configuration and dimension for implantation into a fistula of a patient. Desirably, the coupling structure is configured to engage a placement member (e.g., a wire guide having a loop at one end). Desirably, the coupling structure is configured to be easily attached to a placement member and to remain attached to the placement member while force is exerted on the placement member and attached medical device to properly position the medical device within a patient. The coupling structure may also be configured for easy detachment from the placement member after the medical device is properly positioned within the patient. In some embodiments, the coupling structure comprises a loop, wherein the loop may be, for example, a closed loop or a loop having a discontinuity therein. The coupling structure may comprise a generally convex, concave, S-shaped, straight, curved, flat, polygonal, conical, cylindrical, elliptical, or hemispherical structure, and may further comprise slots, holes, or other openings therein to facilitate engagement with a placement member. For example, in certain embodiments, the coupling structure is a bead-like or button-like structure adapted to engage a placement member, such as a straight wire guide or a wire guide having a loop at one end. In other embodiments, the coupling structure comprises a hook, clamp, clasp, suture, or any other suitable coupling mechanism.

In certain embodiments, the medical device includes an anchoring adaptation to prevent displacement of the medical device and/or its components following implantation of the medical device within a fistula. For example, the medical device may have protrusions on its outer surface to assist in anchoring the medical device within the fistula, or it may have other suitable anchoring adaptations, including but not limited to barbs, hooks, sutures, adhesives, ribs, and the like. Such anchoring adaptations, while advantageous in certain embodiments, are not necessary to broader aspects. Illustratively, certain medical devices are configured so that a cap is used to maintain contact with the tissue adjacent to the primary opening of a fistula following implantation, thereby eliminating the need for such anchoring adaptations, as disclosed in U.S. Publication No. 2007/0031508, published Feb. 8, 2007. In other embodiments, suitable anchoring adaptations may aid or facilitate the maintenance of such contact.

In some aspects, a system for occluding an abnormal bodily passage, such as a fistula, is provided. In certain embodiments, the system comprises an occluding member (e.g., a plug or graft) having a first coupling structure, and a placement member (e.g., a wire guide or similar device) having a second coupling structure. Desirably, the second coupling structure is configured to detachably engage the first coupling structure and to facilitate insertion of the occluding member into the bodily passage. In other embodiments, the first and/or second coupling structures are configured to be trimmed off or otherwise removed from the occluding member body. The first and second coupling structures, which may or may not be the same type of structure, may comprise any suitable structure for coupling the occluding member to the placement member. A few non-limiting examples of such coupling structures are described above.

The body of the occluding member and/or any other components of the medical device of the present invention may have any suitable size and shape for treating bodily openings and passages such as fistulas and may be made of any biocompatible material suitable for implantation into a mammalian body. Desirably, the biocompatible material comprises a biocompatible biological material (e.g., a heterograft, allograft, or autograft material) or a biocompatible synthetic material. More desirably, the material comprises a tissue ingrowth material, which facilitates incorporation of the host tissue of the patient into the body of the occluding member and/or other components of the medical device after implantation. A detailed description of non-limiting illustrative examples of suitable materials for use in the present invention are provided in co-pending U.S. Publication No. 2007/0031508, published Feb. 8, 2007, the contents of which are incorporated by reference. In some embodiments, a sheet form material that is deformable upon impingement by soft tissue is used to form one or more of the components of the medical device. In some embodiments, the material has a collagenous tissue frame that remains intact to allow for ingrowth of host cells and eventual reconstruction of the host tissue itself. Desirable remodelable collagenous materials can be provided, for example, by collagenous materials isolated from a warm-blooded vertebrate, and especially a mammal. Such isolated collagenous material can be processed so as to have remodelable, angiogenic properties and promote cellular invasion and ingrowth. Remodelable materials may be used in this context to promote cellular growth on, around, and/or within tissue in which a medical device of the invention is implanted, e.g., around tissue defining a fistula tract or an opening to a fistula.

Suitable remodelable materials include, but are not limited to, collagenous extracellular matrix (ECM) materials, which are described more fully in co-pending U.S. Publication No. 2007/0031508, published Feb. 8, 2007, the contents of which are incorporated by reference. In some embodiments, naturally-derived ECM materials are used. In other embodiments, synthetic remodelable/regenerative ECM materials are used. The ECM material may be free of additional non-native crosslinking, or may contain additional crosslinking. Examples of suitable collagenous materials include, but are not limited to, ECM materials such as submucosa, renal capsule membrane, dermal collagen, dura mater, pericardium, serosa, peritoneum or basement membrane layers, including liver basement membrane. Suitable submucosa materials for these purposes include, for instance, intestinal submucosa including small intestinal submucosa, stomach submucosa, urinary bladder submucosa, and uterine submucosa. Submucosa useful in certain of the present embodiments can be obtained by harvesting such tissue sources and delaminating the submucosa from smooth muscle layers, mucosal layers, and/or other layers occurring in the tissue source. For additional information as to useful submucosa and its isolation and treatment, reference can be made, for example, to U.S. Pat. Nos. 4,902,508, 5,554,389, 5,993,844, 6,206,931, and 6,099,567, the contents of which are incorporated by reference.

When formed separately, the components of the medical devices may or may not be comprised of the same biocompatible material(s) as the other components of the device. In certain aspects, the components are formed from separate pieces of material, yet are retained in association with one another without the use of any other device or material (e.g., sutures, an adhesive, etc.). For example, the body of the occluding member and the coupling structure may be held together by having at least one member (or any portion thereof) received around, through, over, etc., the other member (or any portion thereof). In some embodiments, a single component of the medical device may comprise one or more types of material. For example, an occluding member body may be made of a multilaminate material comprising a plurality of layers of a single material or of multiple, different materials, where the layers may be bonded together in any suitable manner (e.g., by a bonding agent, cross-linking, or vacuum pressing).

In some embodiments, one or more bioactive agents are included. As used herein, the phrase “bioactive agent” refers to any pharmaceutically active agent that produces an intended therapeutic effect on the body to treat or prevent conditions or diseases. Such bioactive agents may be incorporated into the medical device, coated onto the medical device, or included in the medical device (or portions thereof) in any other suitable manner. For example, a bioactive agent (or a bioactive agent combined with another biocompatible material) may be coated onto the body of the medical device and configured to release over a certain period of time.

Suitable bioactive agents may include one or more bioactive agents native to the source of an ECM tissue material. For example, a submucosa or other remodelable ECM tissue material may retain one or more growth factors including but not limited to basic fibroblast growth factor (FGF-2), transforming growth factor beta (TGF-beta), epidermal growth factor (EGF), cartilage derived growth factor (CDGF), and/or platelet derived growth factor (PDGF). In addition, submucosa or other ECM materials may retain other native bioactive agents including but not limited to proteins, glycoproteins, proteoglycans, and glycosaminoglycans. For example, ECM materials may include heparin, heparin sulfate, hyaluronic acid, fibronectin, cytokines, and the like. Thus, generally speaking, a submucosa or other ECM material may retain one or more bioactive components that induce, directly or indirectly, a cellular response such as a change in cell morphology, proliferation, growth, protein or gene expression.

In addition or as an alternative to the inclusion of such native bioactive components, non-native bioactive components such as those synthetically produced by recombinant technology or other methods (e.g., genetic material such as DNA), may be incorporated into the material used to form the components of certain embodiments of the present medical devices. These non-native bioactive components may be naturally-derived or recombinantly produced proteins that correspond to those natively occurring in an ECM tissue, but perhaps of a different species. These non-native bioactive components may also be drug substances. Illustrative drug substances that may be added to material layers include, for example, anti-clotting agents, e.g. heparin, antibiotics, anti-inflammatory agents, and anti-proliferative agents, e.g. taxol derivatives such as paclitaxel. Such non-native bioactive components can be incorporated into and/or onto a material in any suitable manner, such as by surface treatment (e.g., spraying) and/or impregnation (e.g., soaking), just to name a few non-limiting examples.

Other suitable bioactive agents that may be used include, but are not limited to: antithrombotics, antiplatelets, fibrinolytics, antiproliferative/antimitotic agents, antiplatelet agents, antiproliferative/antimitotic alkylating agents, antiproliferative/antimitotic antimetabolites, platinum coordination complexes, hormones, anticoagulants, fibrinolytic agents, antimigratory agents; antisecretory agents, anti-inflammatory agents, para-aminophenol derivatives, indole and indene acetic acids, immunosuppressives, angiogenic agents, angiotensin receptor blockers, nitric oxide and nitric oxide donors, anti-sense oligionucleotides and combinations thereof, cell cycle inhibitors, retenoids, cyclin/CDK inhibitors, endothelial progenitor cells (EPC), angiopeptin, pimecrolimus, angiopeptin, HMG co-enzyme reductase inhibitors, metalloproteinase inhibitors, protease inhibitors, antibodies, and Liposomal Biphosphate Compounds (BPs). Additional illustrative examples of suitable bioactive agents that may be used in the present invention are set forth in U.S. Publication No. 2007/0031508, published Feb. 8, 2007 and are incorporated herein by reference.

Certain embodiments of the present medical devices may also comprise a variety of synthetic polymeric materials including but not limited to bioresorbable and/or non-bioresorbable plastics. Bioresorbable, or bioabsorbable polymers that may be used include, but are not limited to, poly(L-lactic acid), polycaprolactone, poly(lactide-co-glycolide), poly(hydroxybutyrate), poly(hydroxybutyrate-co-valerate), polygalactin, hyaluronic acid, polydioxanone, polyorthoester, polyanhydride, poly(glycolic acid), poly(D,L-lactic acid), poly(glycolic acid-co-trimethylene carbonate), polyhydroxyalkanaates, polyphosphoester, polyphosphoester urethane, poly(amino acids), cyanoacrylates, poly(trimethylene carbonate), poly(iminocarbonate), copoly(ether-esters) (e.g., PEO/PLA), polyalkylene oxalates, and polyphosphazenes. These or other bioresorbable materials may be used, for example, where only a temporary blocking or closure function is desired, and/or in combination with non-bioresorbable materials where only a temporary participation by the bioresorable material is desired.

Non-bioresorbable, or biostable polymers that may be used include, but are not limited to, polytetrafluoroethylene (PTFE) (including expanded PTFE), polyethylene terephthalate (PET), polyurethanes, silicones, and polyesters and other polymers such as, but not limited to, polyolefins, polyisobutylene and ethylene-alphaolefin copolymers; acrylic polymers and copolymers, vinyl halide polymers and copolymers, such as polyvinyl chloride; polyvinyl ethers, such as polyvinyl methyl ether; polyvinylidene halides, such as polyvinylidene fluoride and polyvinylidene chloride; polyacrylonitrile; polyvinyl ketones; polyvinyl aromatics, such as polystyrene; polyvinyl esters, such as polyvinyl acetate; copolymers of vinyl monomers with each other and olefins, such as ethylene-methyl methacrylate copolymers, acrylonitrile-styrene copolymers, ABS resins, and ethylene-vinyl acetate copolymers; polyamides, such as Nylon 66 and polycaprolactam; alkyd resins; polycarbonates; polyoxymethylenes; polyimides; polyethers; epoxy resins; polyurethanes; rayon; and rayon-triacetate.

Desirably, the biological or synthetic materials used in the present invention assist in reconstruction of the host tissues, elicit little immunological reaction, and have some inherent resistance to infection. Such materials may desirably allow incorporation of the medical device into the host tissue of the fistula (rather than complete absorption of the medical device into the surrounding tissue), thereby occluding the fistula.

The components of the present medical devices (e.g., occluding member body, tail, cap(s), anchoring adaptations, and/or coupling structure), whether formed separately or together as a single unit, can be constructed in any suitable manner. In some embodiments, the occluding member body, tail, cap(s), anchoring adaptations and/or coupling structure are formed with a reconstituted or otherwise reassembled ECM material. Any or all of the components of the medical device may be formed by folding or rolling, or otherwise overlaying one or more portions of a biocompatible material, such as a biocompatible sheet material. The overlaid biocompatible sheet material can be compressed and dried or otherwise bonded into a volumetric shape such that a substantially unitary construct is formed. In some embodiments, a medical device is constructed by randomly or regularly packing one or more pieces of single or multilayer ECM sheet material within a mold and thereafter processing the packed material. Occluding member bodies useful in the present invention can be prepared, for example, as described in U.S. application Ser. No. 11/415,403 (Cook Biotech Incorporated, assignee), the contents of which are incorporated by reference.

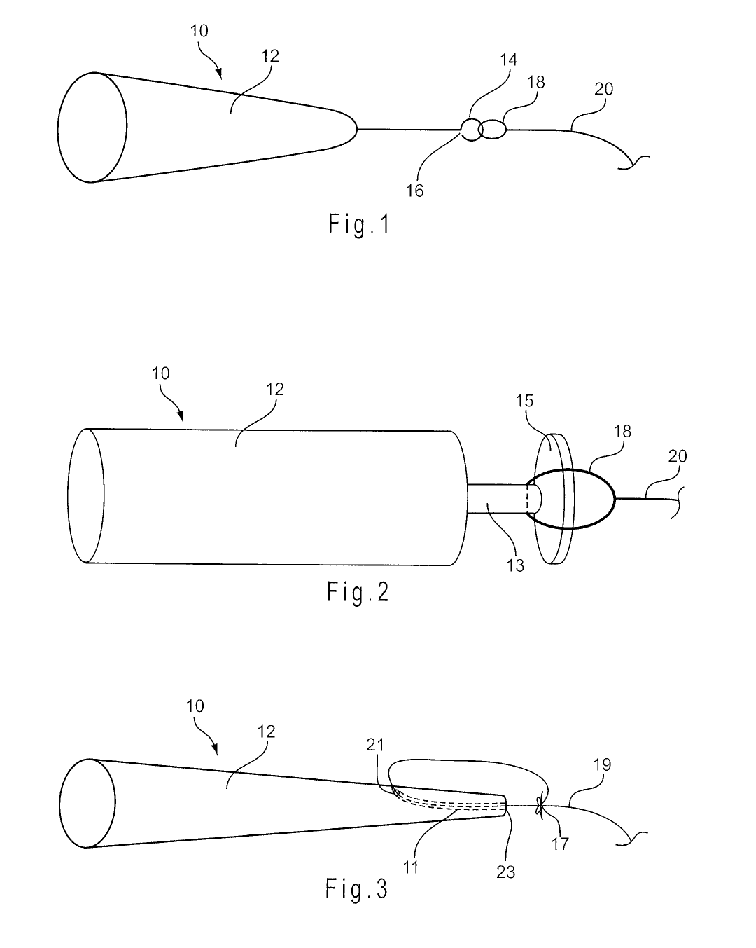

With reference now to the Figures, FIG. 1 shows one embodiment of a medical device 10. In this embodiment, the medical device 10 includes a generally conically shaped occluding member body 12 and a coupling structure comprising a loop 14 having a discontinuity 16 therein. As shown, the coupling structure is configured to engage a loop 18 of a wire guide 20. In one embodiment, the coupling structure is attached near the distal end of occluding body 12. The distal end of occluding member body 12 is the end positioned most distant from the primary opening of the fistula when occluding member body 12 is placed within the fistula so as to block the fistula.

With reference now to FIG. 2, an alternative embodiment of a medical device 10 is shown. In this embodiment, the medical device 10 includes a generally cylindrically shaped occluding member body 12 and a coupling structure comprising an elongate member 13 and a bar member 15. As shown, the coupling structure is configured to engage a loop 18 of a wire guide 20. The bar member 15 functions to prevent the loop 18 of the wire guide 20 from sliding off the end of the elongate member 13 and to keep the medical device 10 attached to the loop 18 of the wire guide 20 so that the medical device 10 can be pulled into a fistula tract, for example, by pulling on the wire guide 20. Although the bar member 15 in this embodiment is shown as having an oblong shape, any suitable shape may be used. Suitable shapes include those that minimize potential trauma to the patient during deployment of the medical device. The elongate member 13 that forms a portion of the coupling structure of this embodiment functions to provide sufficient space between the occluding member body 12 and the bar member 15 to allow the loop 18 of the wire guide 20 to be placed around the elongate member 13 and to contact the bar member 15 so that the bar member 15 can resist the movement of the loop 18 off the elongate member 13 as force is applied to the loop 18 and wire guide 20 during the deployment of the medical device 10.

With reference now to FIG. 3, still another embodiment of a medical device 10 is shown. In this embodiment, the medical device 10 includes a generally conically shaped occluding member body 12 and a coupling structure comprising a lumen 11 extending from an opening 23 at the end of the occluding member body 12 that is adapted to be positioned within or adjacent to the secondary opening of a fistula, through a portion of the occluding member body 12, and terminating at an opening 21 in the exterior surface of the occluding member body 12. In some embodiments, multiple openings may be provided in the exterior surface of the occluding member body. A placement member 19, such as a wire guide, suture, or seton, for example, may be coupled to the medical device 10 by inserting the placement member 19 into either opening 23, 21 and out through the other opening 21, 23. In some embodiments, the end of the placement member 19 extending from the opening 21 in the external surface of the occluding member body 12 is brought around to meet the portion of the placement member 19 extending from the other opening 23 and then secured in any suitable manner. For example, the end of the placement member extending from the opening 21 may be tied or crimped to the portion of the placement member 19 extending from the other opening 23. The embodiment depicted in FIG. 3 illustrates using a knot 17 as a means of securement.

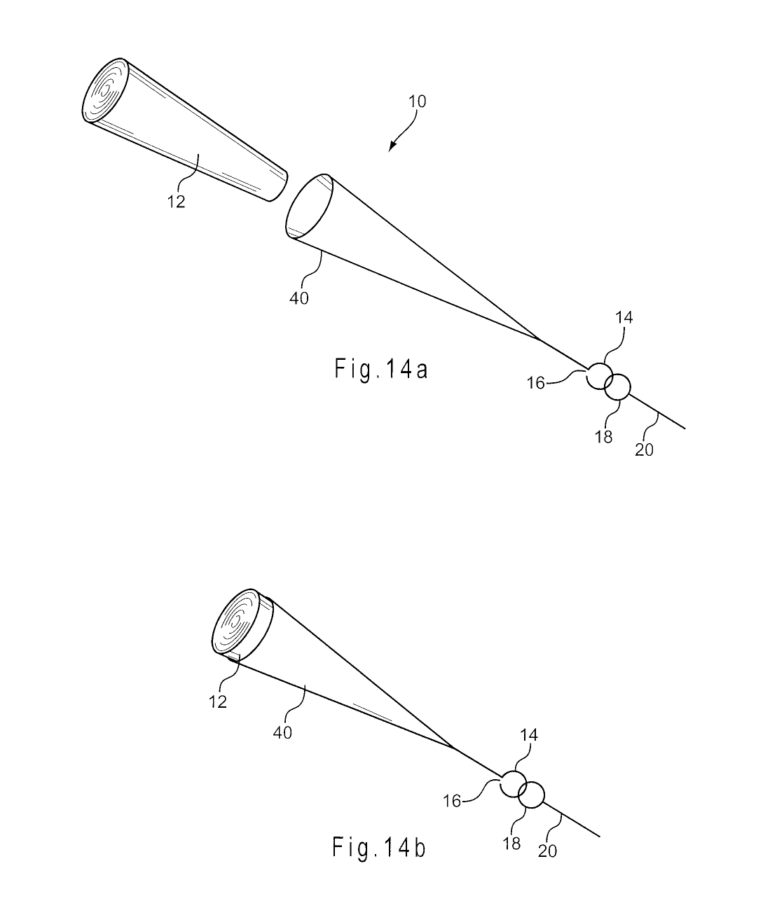

With reference now to FIGS. 14a and 14b, still another embodiment of a medical device 10 is shown. FIG. 14 illustrates an exploded view of medical device 10 including an occluding member having an occluding member body 12 and a sheath 40. Sheath 40 is configured to cover at least a portion of occluding member body 12.

FIG. 14b illustrates a conical occluding member body 12 positioned within sheath 40. However, the occluding body can have other shapes, including but not limited to generally convex, concave, S-shaped, straight, curved, flat, polygonal, conical, cylindrical, elliptical, or hemispherical, or it may have any other configuration capable of being inserted into and secured within a fistula.

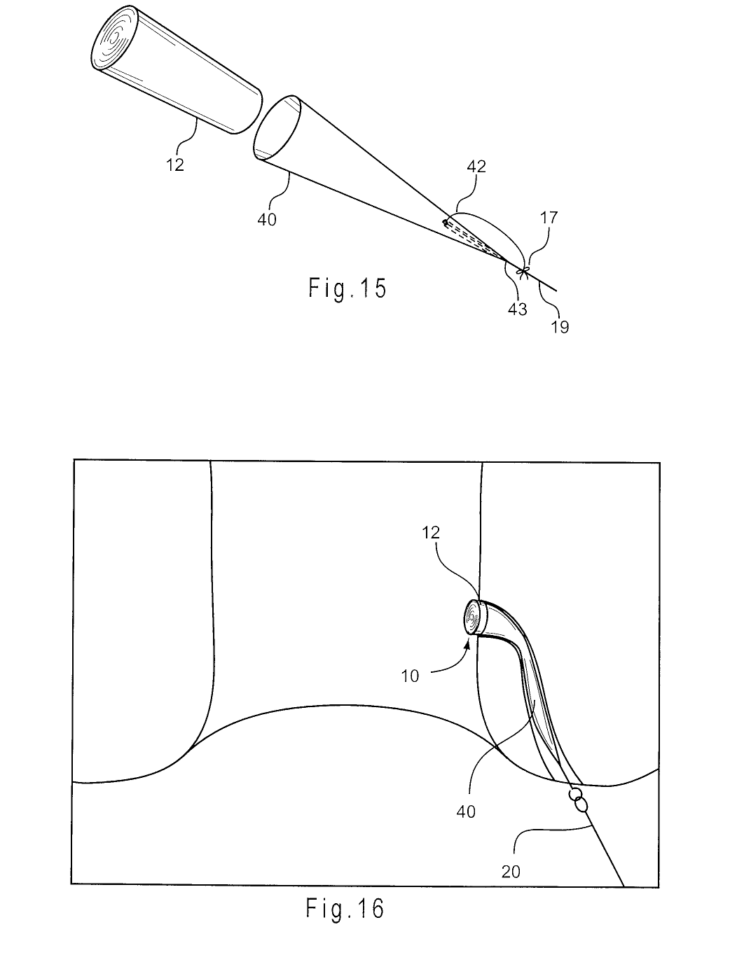

A first coupling structure including loop 14 is attached to sheath 40 and is configured to engage a second coupling structure, including a loop 18, foaming part of a positioning device, such as wire guide 20. In FIGS. 14a and 14b, loop 14 includes a discontinuity 16, whereas loop 18 is a closed loop. In other embodiments, the first coupling structure includes a closed loop or an elongate member and a bar member, such as elongate member 13 and bar member 15 of FIG. 2. FIG. 15 illustrates another embodiment. Here, sheath 40 includes openings 42 and 43. Placement member 19 is coupled to sheath 40 by looping placement member 19 through openings 42 and 43 and securing the end of placement member 19 to another portion of placement member 19 in any suitable manner, such as by knot 17, as is illustrated in FIG. 15.

In one embodiment, sheath 40 is formed from a flexible material such that when sheath 40 is detached from occluding member body 12, the sheath collapses at least partially so as to assist in extraction of the sheath from the fistula. In other embodiments, sheath 40 forms a rigid structure that maintains its shape after separation from occluding body 12. In one embodiment, sheath 40 includes a polymer. In one embodiment, sheath 40 in formed from a material that is at least partially impermeable to fluids such that it protects at least a portion of the occluding body from wetting during placement of the medical device. In other embodiments, the sheath may have a smooth external surface that at least reduces frictional forces during placement of the occluding body within the fistula.

Turning now to a general discussion regarding methods for treating fistulas according to certain of the present embodiments, suitable treatment methods include providing a medical device, such as any of those described herein, and implanting the medical device within a patient so that: (i) the medical device blocks at least the primary opening of a fistula, i.e., the primary opening and potentially one or more other segments of a fistula, for example, the fistula tract and/or any secondary openings; (ii) the cap(s) (if present) contacts portions of the tissues adjacent to the primary opening and/or portions of the tissues surrounding any secondary openings; and (iii) the body of the medical device extends into at least a portion of the fistula tract.

The present medical devices, systems, and methods can be used to treat any fistula, such as a fistula having a primary opening in a wall of an alimentary canal. In some aspects, certain embodiments provide medical devices and methods useful for blocking openings anywhere on or within the body of a patient, for example, blocking at least the primary opening of urethro-vaginal fistulas, vesico-vaginal fistulas, tracheo-esophageal fistulas, gastro-cutaneous fistulas, fistulas occurring between the vascular and gastrointestinal systems, and any number of anorectal fistulas, such as recto-vaginal fistula, recto-vesical fistulas, recto-urethral fistulas, or recto-prostatic fistulas. Also, the present devices and methods can be used to treat a fistula regardless of its size and shape, and in some forms, are used to treat fistulas having a primary opening, secondary opening(s), and/or fistula tract with a diameter ranging from about 1 to about 20 millimeters, more typically from about 5 to about 10 millimeters.

The present medical devices can be implanted using any suitable delivery method or placement technique. Illustratively, an occluding member body can be implanted by pulling the occluding member body into a suitable position within a fistula, either with or without the assistance of additional instrumentation, including but not limited to, catheters, wire guides, probes, scopes, and the like. In certain embodiments, such implantation can be accomplished using a fistula probe or scope or another suitable medical instrument, for example, an appropriately configured pair of surgical hemostats that includes a portion passable into a secondary opening, through the fistula tract, and potentially out of the primary opening. Thereafter, the body of the occluding member can be releasably grasped by the probe or otherwise coupled to the probe and pulled into the primary opening. The body of the occluding member may also be secured at one or both ends by means of sutures, cap(s), or any other suitable method of affixation. In other embodiments, a wire guide and catheter are used to cannulate the fistula, and then the cannulating wire guide is replaced with a second wire guide having a coupling structure at one end. In certain embodiments, a single wire guide may be used to perform the entire procedure, without the need to replace the cannulating wire guide with a second wire guide. In some embodiments, a second wire guide having a stiffness greater than the stiffness of the cannulating wire guide is used. The second wire guide is positioned such that the coupling structure extends out of the primary opening of the fistula. The medical device may then be attached to the coupling structure and pulled into position.

In some embodiments, the medical device includes a tail in association with the body of the occluding member, for example, a tail that is sutured, glued, tied, or attached by another suitable means to the body of the occluding member. This tail can be used to pull the body of the occluding member into a suitable position within a fistula. For example, one end of the occluding member body or the tail of the medical device can be pulled through the primary opening of the fistula and towards the secondary opening until the cap (if present) contacts portions of the tissues adjacent to the primary opening and/or at least a portion of the body of the occluding member becomes wedged into the primary opening. The tail may then be trimmed or removed from the medical device by using, for example, cutting shears. In alternative embodiments, the tail is made from a remodelable or otherwise absorbable material such that it can be left in place within the fistula tract. The tail may be used to anchor or otherwise suitably secure the medical device within the implantation site. For example, the tail can be tied to the tissues of the patient at a suitable location, for example, a location just inside or external to a secondary fistula opening. Further, in alternative embodiments, a medical device can be positioned so that it spans the entire length of a fistula tract, i.e., from the primary opening to a location at or external to a secondary opening. In these embodiments, string or suture, for example, can be used to secure the tail of the medical device to the tissue of the patient at an external location.

In some embodiments, after implanting a medical device into a fistula tract, either end or both ends of the medical device are secured by caps on the medical device, sutures, or other means of securement to ensure that the medical device is not displaced and/or expelled through the primary opening or the secondary opening of the fistula. The suture may be formed as an integral part of the medical device or as a separate component and may be made of any suitable material. Where the medical device includes a cap on the end adapted to be positioned within or adjacent to the primary opening, it may be desirable to secure the other end of the medical device at the level of the primary and/or secondary opening for additional assurance that the medical device will not be displaced or expelled through the primary opening. The use of a cap on each end of the medical device may be desirable to avoid the need for using sutures and piercing the tissues of the patient to firmly secure the medical device within the fistula tract. In some embodiments, at least one cap is expandable so that it can be deployed in an un-expanded position and then expanded after the body of the medical device is properly positioned within the fistula, thereby further securing the medical device within the fistula. In other embodiments, a second cap is attached to the body of the medical device and/or the tissues of the patient using any suitable means of attachment, such as those described herein, after the body of the medical device is properly positioned within the patient.

Fistula treatment methods of the invention may include an endoscopic visualization (fistuloscopy) step, as disclosed in co-pending U.S. Publication No. 2005/0070759A1, published Mar. 31, 2005, hereby incorporated by reference in its entirety. Such endoscopic visualization can be used, for example, to determine the shape and size of the fistula, which in turn can be used to select an appropriately sized and shaped medical device for treating the fistula. Illustratively, a thin flexible endoscope can be inserted into a secondary opening of the fistula and advanced under direct vision through the fistula tract and out through the primary opening. In certain embodiments, a smaller size endoscope, such as a pediatric endoscope (typically under 8 mm in diameter) may be used. By performing fistuloscopy of the fistula, the primary opening can be accurately identified. Also, cleaning of the fistula can be performed prior to and/or during deployment of a medical device of the invention. For example, an irrigating fluid may be used to remove any inflammatory or necrotic tissue located within the fistula prior to implanting the medical device. In certain embodiments, one or more antibiotics are applied to the medical device and/or the soft tissues surrounding the fistula as an extra precaution or means of treating any residual infection within the fistula.

The medical devices of the present invention can be modified before, during, and/or after deployment. Illustratively, the medical device may be cut, trimmed, sterilized, and/or treated (e.g., brought into contact, impregnated, coated, etc.) with one or more desirable compositions, such as any of those disclosed herein, e.g., anticoagulants (e.g., heparin), growth factors or other desirable property modifiers. In certain aspects, following deployment of a medical device in accordance with the present invention, one or more portions of the medical device, for example, material protruding from the primary opening and/or any secondary opening, are trimmed off or otherwise removed.

In certain embodiments, the medical device is anchored within the fistula by threading a securing device having a central lumen, over the tail of the medical device and securing it into position at skin level (e.g., by crimping it). In some embodiments, further anchoring of the medical device is achieved by using a material such as a small intestinal submucosa heterograft (a freeze-dried material that requires rehydration before use) for the medical device and inserting the medical device into the tract before the medical device material has been fully expanded by hydration. In other embodiments, autologous fibrin glue or other suitable adhesive is used in conjunction with the medical device to supplement the adhesive and occlusive properties of the disclosed invention (e.g., Symphony PCS, DePuy AcroMed Inc.).

Closure of a fistula tract may be performed as a one-stage or two-stage procedure. As a one-stage procedure, the fistula tract is closed or sealed at the same time as the initial surgery. As a two-stage procedure, a seton (which may be incorporated as a part of the medical device) is first placed through the fistula tract to allow mechanical drainage of the fistula tract and to mature the fistula tract prior to a definitive closure procedure. The seton may be passed through the fistula tract and tied as a loop around the contained tissue and left for several weeks or months. Subsequently, the seton may be removed and the medical device inserted into the fistula. In certain embodiments of the method of the present invention, a tail associated with the body of the occluding member is used to eliminate the seton placement step.

In some aspects, where multiple fistulas are present, multiple medical devices may be inserted until all fistula tracts have been closed. In the case of a complex fistula, for instance a horseshoe fistula, there may be one primary opening and two or more tracts leading from that opening. In this instance, a medical device may be configured with one proximal end (e.g., a larger diameter end), and two distal ends (e.g., smaller diameter ends). Desirably, accurate identification of all fistula tracts and the primary opening is facilitated by first performing fistuloscopy. Once the tracts have been identified and cleaned out, each distal end may be pulled through the primary opening into each fistula in turn, desirably using the instruments and methods disclosed herein. Adequate force is applied to the medical device and/or associated placement member to ensure that the proximal end of the body of the medical device is firmly secured in the primary opening of the fistula and/or the cap (if present) attached to the proximal end of the body of the medical device contacts the tissues adjacent to the primary opening. The proximal end of the medical device and/or each of the tails (if present) may be further secured by any suitable means of securement, including but not limited to those described above.

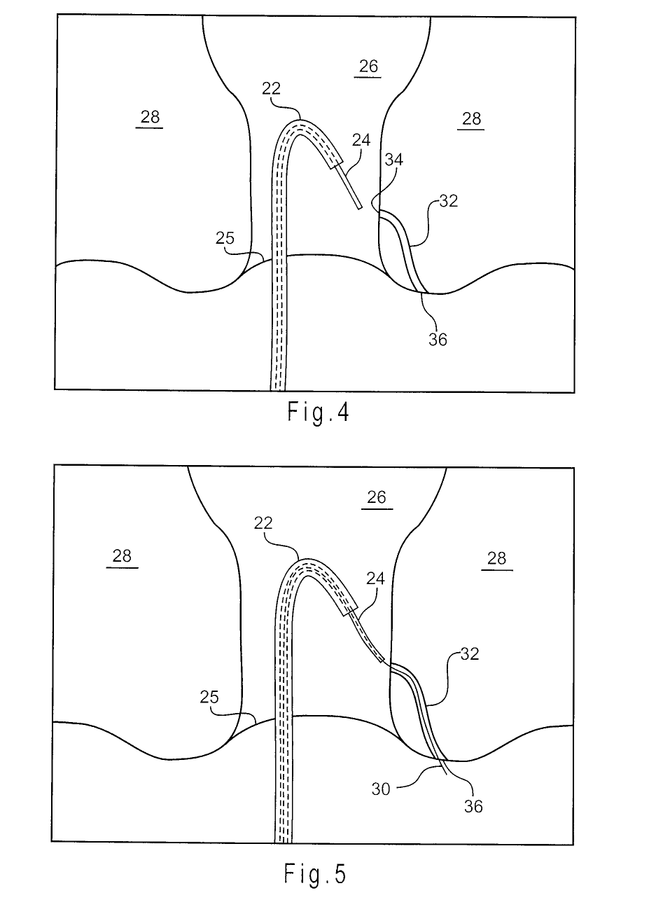

With reference now to FIGS. 4 through 12, one embodiment of a method of placing an occluding member within a fistula is depicted. In this embodiment, the method involves occluding an anorectal fistula within a patient. While these Figures illustrate the treatment of an anorectal fistula, it will be understood that the present devices, systems, and methods may be useful in treating other types of fistulas as well, and in some embodiments, are useful in occluding, filling, blocking, or otherwise treating non-fistula openings or passages occurring in the body.

As shown in FIG. 4, the primary opening 34 of the fistula tract 32 may be located within the tissues surrounding the rectum 26, near the dentate line 25 of the patient. The secondary opening 36 of the fistula tract 32 is located in the perianal skin on the buttock 28 of the patient. In this embodiment, an endoscope 22 is positioned within the rectum to facilitate visualization of the fistula tract 32. In some embodiments, the endoscope 22 is placed in a retroflex position. A catheter 24 may then be placed within an instrument channel of the endoscope 22. The distal end of the catheter 24 is extended distally from the instrument channel of the endoscope 22 and positioned in close proximity to the primary opening 34 of the fistula tract 32. In desirable embodiments, a catheter having a size of about 1-10 french is used, and more desirably, a catheter having a size of about 4-5 french is used. The catheter desirably has a single lumen and is about 130-165 cm in length (more desirably 145-150 cm in length), but any other suitable catheter may be used. A dome tipped catheter may be desirable to minimize trauma to the patient.

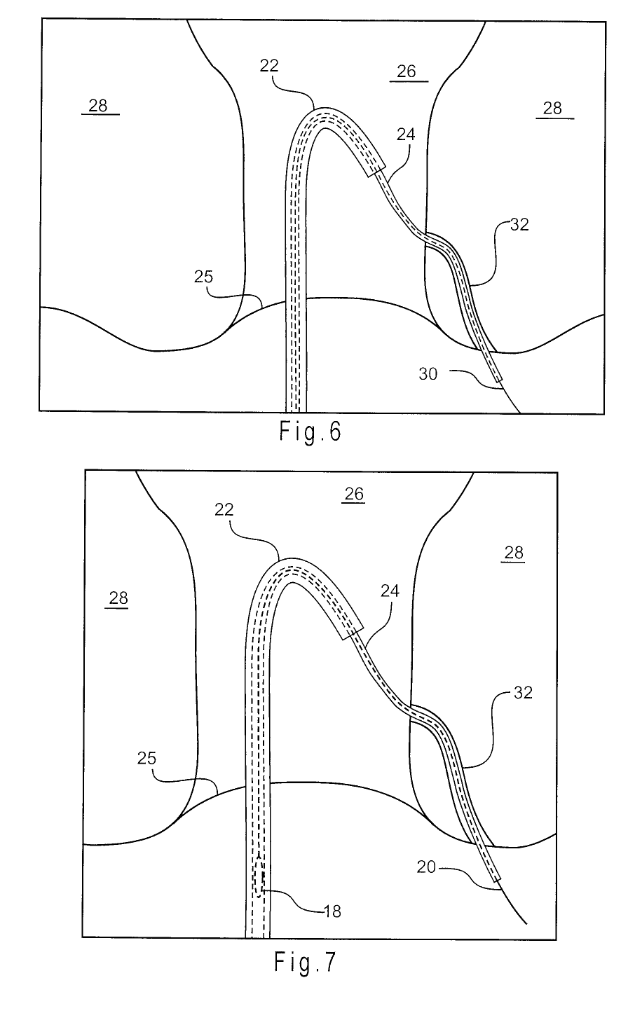

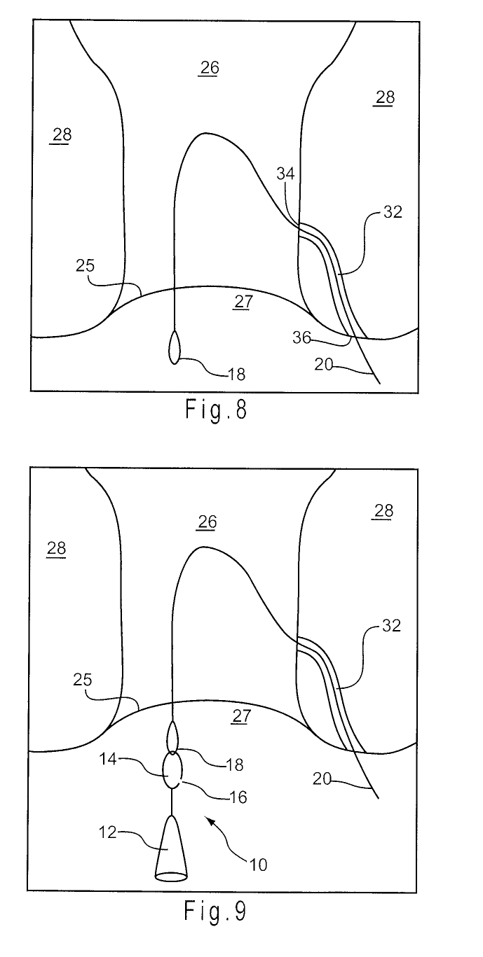

As shown in FIG. 5, a wire guide 30 may then be inserted through the lumen of the catheter 22, through the primary opening, and into the fistula tract 32. The wire guide 30 may then be advanced through the fistula tract 32 and out through the secondary opening 36. Desirably, the wire guide 30 is a cannulating wire (more desirably, a cannulating wire having a hydrophilic soft floppy tip), but any suitable type of wire guide may be used. As shown in FIG. 6, the catheter 24 may then be advanced over the wire guide 30 and into the fistula tract 32. The wire guide 30 may then be removed and a placement member, such as a wire guide 20 having a coupling structure at the proximal end thereof, such as a loop 18, may then be inserted through the catheter 24, as shown in FIG. 7. Alternatively, the step of inserting the cannulating wire guide can be eliminated and the second wire guide having a coupling structure at the proximal end thereof can be inserted through the catheter in the first instance. Next, the catheter 24 and endoscope 22 may be removed from the patient (while holding onto the distal portion of the wire guide 20 positioned outside the secondary opening, for example), leaving the central portion of the wire guide 20 positioned through fistula tract 32, as shown in FIG. 8. At this stage of the procedure, the wire guide 20 extends from outside the secondary opening 36, through the fistula tract 32, out through the primary opening 34, through the rectum 26, and out through the anal canal 27 of the patient. As shown, the loop 18 of the wire guide 20 is now positioned outside the patient.

As shown in FIG. 9, the medical device 10 may then be coupled to the coupling structure 18 of the wire guide 20. The method of coupling a medical device to a placement member, such as a wire guide or a string, varies depending upon the coupling structure of the medical device and the coupling structure of the placement member. For example, if the coupling structure of the medical device comprises a loop 14 having a discontinuity 16 therein and the coupling structure of the placement member is a closed loop 18, as shown in FIG. 9, the two devices may be coupled together by simply inserting the loop 18 of the placement member 20 through the discontinuity 16 in the loop 14 of the medical device 10. The discontinuity 16 in the loop 14 of the medical device 10 may then be closed, for example by squeezing the loop 14 together until the ends overlap, to prevent the loop 18 of the placement member 20 from inadvertently slipping out through the discontinuity 16. Alternatively, for a medical device 10 such as the one depicted in FIG. 2, where the coupling structure comprises an elongate member 13 and a bar member 15, the medical device 10 may be coupled to a placement member, such as a wire guide 20 having a loop 18, by placing the loop 18 of the wire guide 20 over the bar member 15 and around the elongate member 13, as shown in FIG. 2. In this embodiment, the bar member 15 prevents the loop 18 of the wire guide 20 from detaching from the coupling structure of the medical device 10. Numerous other coupling arrangements are possible. For example, hooks, clamps, clasps, sutures, or any other suitable coupling mechanism may be used in the present invention.

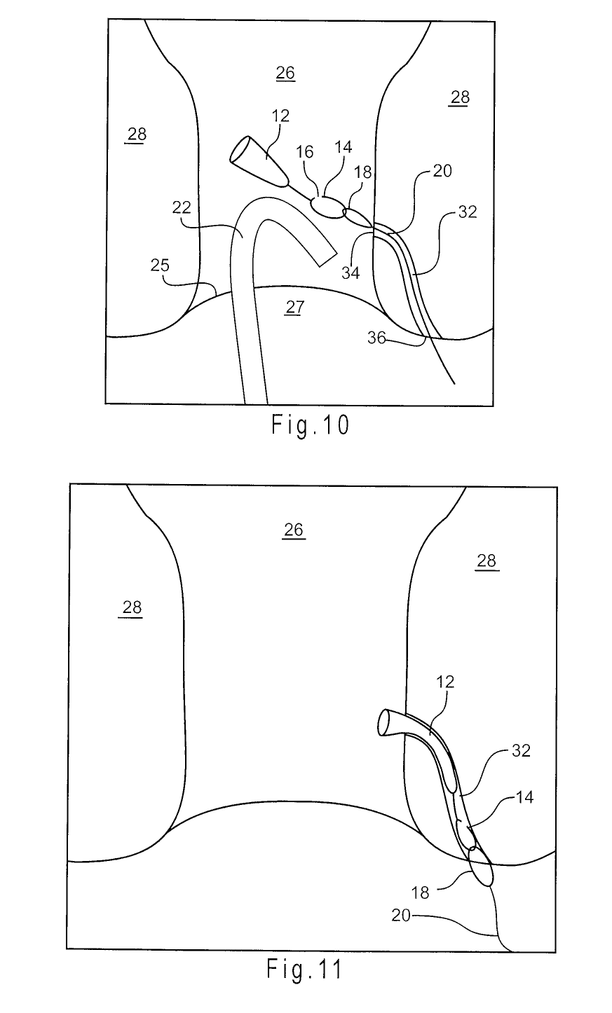

After the medical device 10 is coupled to the placement member 20, the wire guide 20 may be pulled through the fistula tract 32, thereby maneuvering the medical device closer to the primary opening 34, as shown in FIG. 10. The body 12 of the medical device 10 can be advanced through the fistula tract 32 in any suitable manner, and in some embodiments, is pulled through the fistula tract 32 by grasping the wire guide 20 with a grasping device (not shown) such as surgical hemostats, snare, forceps, or a human hand, for example, and pulling the body 12 of the medical device 10 into position. At this stage of the procedure, it may be advantageous to insert an endoscope 22 into the rectum 26 and/or anal canal 27 of the patient to facilitate visualization of the fistula tract 32 and placement of the medical device 10 therein. As shown in FIG. 11, the wire guide 20 and the medical device coupled thereto are pulled into the fistula tract 32 until the body 12 of the medical device 10 contacts the inner wall of the fistula and becomes sufficiently secured within the fistula tract 32. The placement member 20 may then be decoupled from the medical device in any suitable manner.

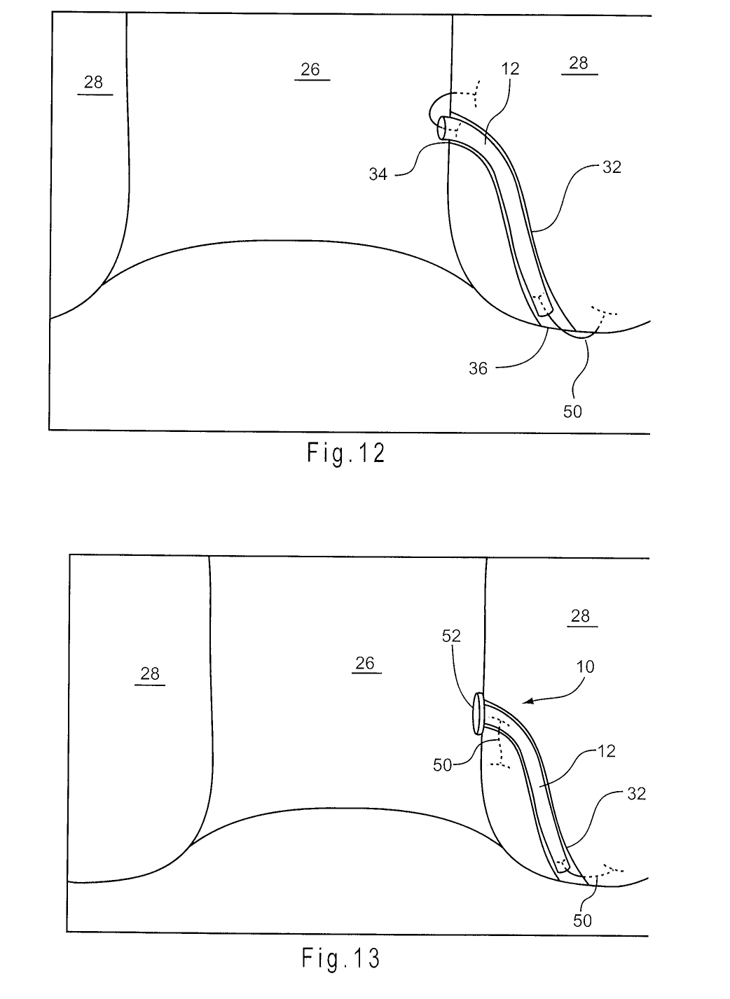

After the medical device is secured within the fistula tract, each end of the device may be trimmed to prevent any excess portions from protruding from the primary and/or secondary openings of the fistula after the procedure. As shown in FIG. 12, the portion of the medical device adjacent the secondary opening 36 has been trimmed and an anchoring member 50, such as a T-fastener, has been used to further secure the medical device within the fistula. In certain embodiments, the portion of the medical device that is shown in FIG. 12 as protruding from the primary opening 34 may also be trimmed so that it is flush with the primary opening 34.

In various embodiments, an anchoring member 50 may be used to secure the medical device at the primary opening or/and the secondary opening of the fistula. In one embodiment, an anchoring member secures the medical device at the primary opening of the fistula. Any suitable anchoring mechanism may be used on one or both ends of the medical device, including but not limited to T-fasteners, caps, barbs, hooks, sutures, adhesives, and ribs, just to name a few non-limiting examples. In some embodiments, an anchoring mechanism is not necessary to firmly secure the medical device within the fistula tract.

With reference now to FIG. 13, an alternative embodiment of medical device 10 is shown implanted within a fistula tract 32. In this embodiment, the medical device includes a generally cylindrical occluding member body 12 and a cap 52. The cap 52 may be used to better secure the occluding member body 12 within the fistula tract 32 and to prevent the occluding member body 12 from being displaced through the primary opening during exercise, exertion, or straining by the patient. The cap 52 may be permanently attached to the occluding member body 12 or it may be configured to detach from the occluding member body 12 after a certain period of time sufficient for the occluding member body 12 to become ingrown into the fistula tract 32, as described in co-pending U.S. Publication No. 2007/0031508, published Feb. 8, 2007, which is hereby incorporated by reference in its entirety. An additional anchoring member 50 may also be used to further secure the occluding member body 12 within the fistula tract 32 at the primary opening or/and the secondary opening, as shown in FIG. 13. In some embodiments, such an anchoring member is not necessary to assure that the occluding member body is adequately secured within the fistula. In other embodiments, a second cap is used as an anchoring member. As explained in co-pending U.S. Publication No. 2007/0031508, published Feb. 8, 2007, the contents of which are incorporated by reference, the cap may be expandable or non-expandable and may be adjustable to various positions along the body of the occluding member. In certain embodiments, a first cap is integral with or otherwise associated with the end of the occluding member body that is adapted to be placed in or adjacent to the primary opening of a fistula, and after deployment of the occluding member body and cap, a second cap is attached to the end of the occluding member body that is adapted to be placed in or adjacent to the secondary opening, thereby securely anchoring the occluding member within the fistula.

In certain other embodiments, occluding member body 12 is shaped so as to block the primary opening of the fistula and that portion of the fistula tract near to the primary opening but to leave a space between occluding body 12 and the fistula wall in the region of the secondary opening. The presence of such a space can assist in allowing for drainage of the fistula.

In certain embodiments, the present medical device is used in conjunction with a sealant or sclerosing solution which may be injected into the main fistula tract and any side branches. Several possible sealants are described in the prior art. One of the more commonly used sealants is fibrin glue, known as Tisseal (Baxter Inc.).

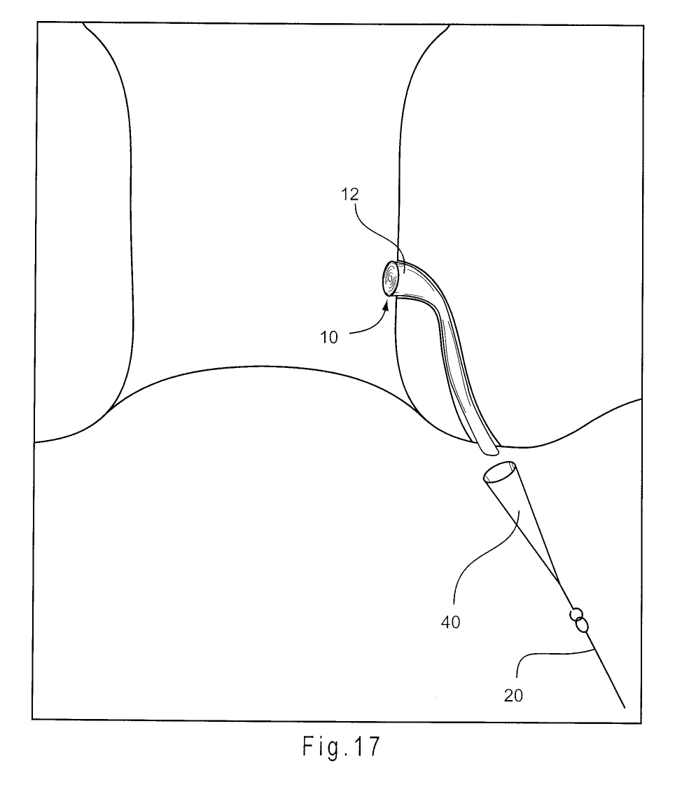

With reference now to FIGS. 16 and 17, another embodiment of a method of occluding a fistula within a patient is depicted. In this embodiment, medical device 10 includes occluding member 12 and sheath 40, such as the devices illustrated in FIG. 14 or 15 and discussed above. Occluding member 12 is positioned within the fistula tract by any of the procedures illustrated above. Upon securing occluding member 12 within the fistula track, sheath 40 is detached from occluding member 12 and removed from the fistula tract by pulling on guide wire 20. In certain embodiments, occluding member 12 is wedged in the fistula tract before sheath 40 is detached and removed from the fistula tract. In other embodiments, occluding member 12 is secured by an anchoring member, such as a T-fastener, before sheath 40 is detached and removed from the fistula tract.

All publications and patent applications cited in this specification are hereby incorporated by reference in their entirety, as if each individual publication or patent application were specifically and individually indicated to be incorporated by reference. Further, any theory, mechanism of operation, proof, or finding stated herein is meant to further enhance understanding of the present invention, and is not intended to limit the present invention in any way to such theory, mechanism of operation, proof, or finding. While the invention has been illustrated and described in detail in the drawings and foregoing description, the same is to be considered as illustrative and not restrictive in character, it being understood that only selected embodiments have been shown and described and that all equivalents, changes, and modifications that come within the spirit of the inventions as defined herein or by the following claims are desired to be protected.

We claim:

1. A method of occluding a fistula having an interior wall, a primary opening, a secondary opening, and a fistula tract, the method comprising:
inserting a first end of a placement member into the primary opening and at least partially into the fistula tract, wherein the placement member comprises a thin, elongated member comprising a second coupling structure at a second end;
detachably connecting the second coupling structure to an occluding member comprising an occluding member body and a first coupling structure, wherein the first coupling structure is connected to the second coupling structure; and
inserting the occluding member into the fistula by pulling the placement member through the fistula until the occluding member contacts the interior wall of the fistula.

2. The method of claim 1 wherein at least one of the first coupling structure and the second coupling structure comprises a loop having a discontinuity therein.

3. The method of claim 2 wherein one of the first coupling structure and the second coupling structure comprises a closed loop.

4. The method of claim 1, wherein the occluding member further comprises a detachable sheath covering at least a portion of the occluding member body, and wherein the first coupling structure is attached to the sheath.

5. The method of claim 4, wherein pulling the placement member through the fistula comprises pulling the placement member so as to detach the sheath from the occluding member body and to extract the sheath from the fistula.

6. The method of claim 1, wherein the occluding member body further comprises a lumen extending from a first opening in the occluding member body, through a portion of the occluding member body, to a second opening in the occluding member body and wherein the placement member comprises a wire guide having a first end and a second end, and the second coupling structure comprises a portion of the wire guide adjacent to the first end.

7. The method of claim 6, further comprising inserting the first end of the wire guide into the first opening of the occluding member body, through the lumen of the occluding member body, and out through the second opening of the occluding member body, and securing the placement member to the occluding member body by tying the first end of the wire guide to a portion of the wire guide extending from the first opening of the occluding member body.

8. The method of claim 1 wherein the fistula is an anorectal fistula.

9. The method of claim 1 further comprising using an endoscope to assist with inserting the placement member into the fistula.

10. The method of claim 1, further comprising anchoring the occluding member within the fistula.

11. The method of claim 10, wherein anchoring the occluding member within the fistula comprises securing the occluding member near the primary opening of the fistula.

12. The method of claim 1, wherein inserting the placement member into the primary opening and at least partially into the fistula tract comprises
inserting a wire guide into the primary opening and at least partially into the fistula tract;
placing a catheter over the wire guide and advancing the catheter at least partially into the fistula tract;
removing the wire guide from the catheter and the fistula tract;
inserting the placement member into the catheter and advancing the placement member at least partially into the fistula tract; and
removing the catheter from the fistula tract.

13. The method of claim 12, wherein the step of inserting the wire guide into the fistula tract comprises first inserting the wire guide into an instrument channel in the endoscope.

14. The method of claim 1, wherein the occluding member body comprises an extracellular matrix material.

15. The method of claim 14, wherein the extracellular matrix material is selected from the group consisting of submucosa, renal capsule membrane, dermal collagen, dura matter, pericardium, serosa, peritoneum and basement membrane layers.

16. The method of claim 14, wherein the extracellular matrix material comprises a delaminated submucosa layer.

17. The method of claim 14, wherein the extracellular matrix material is a remodelable sheet form material.

18. The method of claim 17, wherein the extracellular matrix material remains intact to allow for ingrowth of host cells and restruction of host tissue.

19. The method of claim 14, wherein the extracellular matrix material retains a native bioactive agent.

20. The method of claim 1, wherein the occluding member body comprises a synthetic polymeric material.
